(19) United States
(12) Patent Application Publication
(10) Pub. No.: US 2017/0266009 A1
(43) Pub. Date: Sep. 21, 2017

(54) Full-Ceramic Resurfacing Prosthesis Having a Porous Inner Face

(71) Applicant: CeramTec GmbH, Plochingen (DE)

(72) Inventors: Roman PREUSS, Kirchheim unter Teck (DE); Marita RASCHKE, Leonberg (DE); Peter STRÖTGEN, Königsfeld (DE); Tobias WEISS, Ebersbach (DE)

(21) Appl. No.: 15/324,532

(22) PCT Filed: Jul. 1, 2015
(86) PCT No.: PCT/EP2015/064925

§ 371 Date: Apr. 26, 2017

(30) Foreign Application Priority Data
Jul. 9, 2014 (DE) 10 2014 213 361.0

(51) Int. Cl. A61F 2/36; A61L 27/54; A61L 27/56; A61L 27/10

(52) U.S. Cl. CPC A61F 2/36; A61L 27/10; A61L 27/54; A61L 27/56; A61F 2002/3093

(57) ABSTRACT

The invention relates to a resurfacing prosthesis for the natural femoral head, having a spherical outer surface and an inner receiving space that has an inner surface facing the bone. In order to be able to make the resurfacing prosthesis without using metals, combine the advantages of bone-preserving surgery with those of a biocompatible material, keep wear of the sliding pair low, and anchor the prosthesis in an excellent and long-term stable manner in the bone, the resurfacing prosthesis is characterized by an integrated porous osseointegrative inner surface and by an entirely ceramic design of the resurfacing prosthesis.

The invention relates to a resurfacing prosthesis for the natural femoral head having an outer spherical surface and an inner receiving space with an inner lateral surface facing the bone.

The current clinical prior art, for wear of the natural femoral head or hip joint head, provides a resurfacing prosthesis made of metal or ceramic material. Ceramic resurfacing prostheses to date are largely based on known metallic designs, and are basically cemented. The inner prosthesis surface is provided with cement pockets and a centering pin. Owing to an exothermic reaction, which occurs during polymerization of the bone cement, there is a danger that the adjacent bone tissue may be damaged by heat. Further, the bond between the ceramic material and bone cement is not stable over the long term, and after a few years there may be loosening of the prosthesis resulting in failure of the implant.

EP 0 542 815 B1, EP1 268 364 A1, and DE 10 2013 202 243 A1 are the prior art.

A cementless, full ceramic resurfacing prosthesis according to the invention with integrated porous osseointegrative inner lateral surface constitutes an advantageous novelty.

Such a metal-free resurfacing prosthesis combines the advantages of the bone-sparing operation with those of a biocompatible material, low wear of the sliding pair, and excellent anchoring in the bone with long-term stability.

A resurfacing prosthesis according to the invention for the natural femoral head with an outer spherical surface and an inner receiving space with inner lateral surface is characterized by an integrated porous osseointegrative surface and by a full ceramic design. The advantages of this resurfacing prosthesis are further described above.

A method according to the invention for manufacturing the integrated porous osseointegrative surface of the resurfacing prosthesis according to claim 1 is characterized in that the resurfacing prosthesis is made of a sintered molded article and that a porous osseointegrative layer is applied to the surface. Lateral surface is understood to be the face facing the bone, which lies against the bone after implantation.

In an embodiment of the method according to the invention, the porous lateral surface is produced by application of a ceramic slurry and place-holders (pore-formers) in the green state and burning off of the place-holders during sintering. Preferably, the porous layer can be created by foaming of a ceramic slurry in the green state and subsequent sintering. A bond is achieved by means of the sintering process. By burning off of the place-holders during sintering, an open-pored structure is produced with a roughened surface, which advantageously supports the accretion and ingrowth of the bone. See EP 1 268 364 A1 also in this regard.

The porous lateral surface can also be created by joining of a dense molded article with a porous ceramic molded article in the sintered state. The porous lateral surface can be created by foaming of a ceramic slurry and sinters or infiltration of a porous substrate material with slurry and sinters. The joining is preferably carried out by means of soldering or gluing.

By means of the sintering method, a bond is achieved and an open-pored structure with a roughened surface is created that advantageously supports the accretion and ingrowth of the bone. This type of coating in combination with a ceramic main body is advantageously metal-free with respect to allergic reactions.

The porous osseointegrative lateral surface according to the invention can also be produced by 2c-injection molding (dense phase and porous or pore-forming phase) and sintering. This type of coating in combination with a ceramic body is also metal-free.

In a further embodiment according to the invention, the integrated porous osseointegrative lateral surface is created by application of a porous metallic biocompatible layer on the already sintered ceramic main body (see EP 1 052 949 B1).

The porous lateral surface facing the bone through further coating can acquire an additional functionality, such as higher integration speed or antibacterial effects. Integration speed is understood as the time required for ingrowth with the bone. Preferably this can be achieved with bioglasses, hydroxylapatite, functionalized proteins, or hydrogels. Preferably this can be achieved with bioglasses, hydroxylapatite, functionalized proteins, or hydrogels.

The resurfacing prosthesis according to the invention consists entirely of a ceramic and can be implanted directly through the porous lateral surface. The bone grows quickly into the porous lateral surface, so that a firm seat is ensured quickly after the operation. No bone cement is used in the implantation.

1. A resurfacing prosthesis for the natural femoral head having an outer spherical surface and an inner receiving space with an inner lateral surface facing the bone, characterized by an integrated porous osseointegrative lateral surface and by a full ceramic resurfacing prosthesis.

2. A method for manufacturing the integrated porous osseointegrative lateral surface of the resurfacing prosthesis according to claim 1, wherein the resurfacing prosthesis is produced from a sintered molded article and that a porous osseointegrative layer is applied to the lateral surface.

3. The method according to claim 2, wherein the porous lateral surface is produced by application of a ceramic slurry and place-holders (pore-formers) in the green state and burning off of the place-holders during sintering.

4. The method according to claim 2, wherein the porous lateral surface is produced by foaming of a ceramic slurry in the green state and subsequent sintering.

5. The method according to claim 2, wherein the porous lateral surface is created by joining of a dense molded article with a porous ceramic molded article in the sintered state.

6. The method according to claim 5, wherein the porous molded article is produced by foaming of a ceramic slurry and sinters or infiltration of a porous substrate material with slurry and sinters.

7. The method according to claim 5, wherein the joining is carried out by soldering or gluing.

8. The method according to claim 2, wherein the porous lateral surface is produced by 2c-injection molding (dense phase and porous or pore-forming phase) and sintering.

9. The method according to claim 2, wherein the porous lateral surface is created by application of a porous metallic biocompatible layer on the already sintered resurfacing prosthesis.

10. The method according to claim 2, wherein through further coating the porous lateral surface acquires an additional functionality, such as higher integration speed or antibacterial effects.

11. The method according to claim 10, wherein the porous rear face is coated with bioglasses, hydroxylapatite, functionalized proteins, or hydrogels.
